(19) United States
(12) Patent Application Publication
(10) Pub. No.: US 2004/0087833 A1
(43) Pub. Date: May 6, 2004

(54) Retractor

(76) Inventors: Thomas Bauer, Nehren (DE); Carsten Geister, Tuttlingen (DE); Steffen Kernbach, Bosingen (DE); Gerd Haimerl, Villingen-Schwenningen (DE); Sara Lozano, Villingen-Schwenningen (DE); Norbert Gut, Donaueschingen (DE)

Correspondence Address: NATH & ASSOCIATES PLLC, 6th Floor, 1030th 15th Street, NW, Washington, DC, 20005, US

(21) Appl. No.: 10/364,652

(22) Filed: Feb. 12, 2003

(30) Foreign Application Priority Data
Oct. 30, 2002 (DE) 202 16 712.7

(51) Int. Cl. A61B 1/32

(52) U.S. Cl. 600/201

(57) ABSTRACT

The invention relates to a retractor (10) with a carrier ring (20) and L-shaped valves (30), which can be attached with an arm (32) which runs parallel to the carrier ring plane to the carrier ring (20), whereby its free end points outward, and whose other arm (34) is oriented perpendicularly with a respect to the carrier ring plane and presents, in cross section, a convex curvature toward the external margin of the carrier ring (20), where the arm of (32) [sic; arm (32)] of the valves (30), which is attached to the carrier ring (20), is guided in radically oriented guides (22) on the carrier ring (20).

The invention concerns a retractor, in particular for the treatment of the lateral lumbar intervertebral disc prolapse through the posterolateral access.

Because of the possibility of modern diagnostic imaging lateral intra- or extraforaminal intervertebral disk prolapse can be reliably diagnosed as a cause of root compression symptomatics. Since the site of the lesion is distal with a respect to the nerve root sheath, these changes could not be demonstrated by myelography in the past. In the first surgical treatments, the surgical access was medial via an osteoclastic laminotomy, with partial resection of the joint facets. Special surgical problems resulted from the fact that, because of the distal site of the lesion with a respect to the nerve root sheath, the site can be nearly completely covered by osseous structures of the intervertebral joint so that, if one proceeds from the intraspinal position, large sections of the joint or even both facets must be completely resected in order to allow the precise localization, mobilization, extirpation of the sequestrum and to allow adequate decompression of the nerve root. This entails the risk of an extended postoperative cicatrisation in the sense of an epidural fibrosis as well as, in addition, the possibility of a later instability of the segment.

The posterolateral microsurgical access constitutes an alternate surgical access. Here an incision is made into the paraspinal skin and fascia, and the transverse processes of the vertebral bodies of the affected segment are exposed through the muscles, the intertransverse space is opened using the microsurgical technique, and the nerve root is exposed in an extraforaminal position. The drawback of this surgical treatment is that any lateral portions of the vertebral joint, must, in addition, be milled. In the process, during the access, viewing in the medial direction for evaluating the exact conditions in the neuroforamen is limited. The long transmuscular access is also frequently disadvantageous, as it requires a considerably longer incision of the skin and, in spite of the surgical microscope, it makes the preparation in the depth of the intertransverse space considerably more difficult.

However, the advantages of the posterolateral access compared to the medial access are obvious because, on the one hand, the spinal canal does not need to be opened, and in addition, there is no risk of damaging the epidural space or of a postsurgical epidural fibrosis; moreover, the extent of the required resection of the facet for the inspection of the extraforaminal pathology is reduced and consequently the risk of postoperative instabilities is minimized.

The problem of the invention is to optimize the surgical treatment of the lumbar intervertebral disk prolapse in a lateral position by using the posterolateral access. In the process, one should strive, in particular, for a reduction of the traumatization of the tissue in the area of the access, in particular the muscles of the back, an improvement of the preparative possibilities and the safety in the intertransverse space, and improved visualization of structures which are in a medial position in the neuroforamen without additional bone resection.

The problem is solved by a retractor with the characterizing features of claim 1.

Advantageous developments and executions of the retractor are indicated in the dependent claims.

In the retractor according to the invention, with a carrier ring and L-shaped valves which can be attached to the carrier ring by means of an arm which is parallel to the carrier ring plane, the arm of the valves which is attached to the carrier ring is guided in radially oriented guides on the carrier ring. This allows a controlled movement of the valves of the retractor. When the valves move, they can be moved exclusively in the radial direction, and a rotation of the valve about the point of attachment to the carrier ring is not possible. Thus, unnecessary damage to the tissue enclosing the surgical field is prevented.

In an advantageous execution of the invention, the arch of the valve is designed such that, during the shifting of the valves radially from inside to the middle point of the carrier ring, a complete tube forms, consisting of the arms of the valves which are oriented perpendicularly with respect to the carrier ring plane. In an advantageous execution of the invention, four valves which are evenly distributed on the carrier ring form a complete tube. The complete tube can easily be positioned in a targeted manner in the surgical field in order to shift the valves outward onto the intervertebral disks and to thus increase the accessible area of the surgical field.

Advantageously, each valve can be fixed in the guide by means of a screw which can be regulated by hand. Thus, on the one hand, all the valves can be moved independently of each other, and, on the other hand this arrangement allows a continuous adjustment of the valve and stoppage in any desired position.

An additional advantageous development of the invention presents valves of different lengths attached to the carrier ring. This allows a varied range of applicability of the retractor. If the surgical field is limited by a vertebral joint, a different dimension of the length of the valve offers the possibility of bracing a shorter valve on the vertebral joint and uncovering the lateral surgical field by means of two longer valves on the side of the vertebral joint of the surgical field.

It is preferred for additional guide grooves to be sunk into the carrier ring of the retractor, in which slides are inserted to which surgical instruments such as, for example, endoscopes or similar devices can be attached. This allows a controlled fixation of the surgical instrument in any position. It is preferred to insert a bolt vertically on a slide, to which bolt a horizontal chamber is attached by means of a knurled nut, in which the endoscope or additional instruments are attached.

To facilitate the introduction of the retractor into the muscles, in an advantageous execution of the invention, a handle can be attached to the carrier ring for positioning the retractor. After the introduction of the retractor by means of valves which are telescoped by means of the handle to form a tube, the handle can be removed, so that, subsequently, the instruments are attached to the intervertebral disk and the valves can be shifted away from each other.

It is advantageous for all the components of the retractor to be made of titanium. This material is not reactive, and thus does not attack the tissue.

The retractor according to the invention is used as follows for the treatment of a lateral lumbar intervertebral disk prolapse. The access to the site of the lesion starts at an incision into the skin, whose location is 7 cm paravertebral, and dorsal, and it goes through the muscles in a ventral medial direction, toward the transverse processes of the adjacent vertebrae and corresponding small vertebral joint. As a function of the thickness of the subcutaneous fat layer and the dorsal muscles, the length of the access route is approximately 8-12 cm. The surgical field is limited, cranially and caudally, by the transverse processes of the adjacent vertebrae, and medially by the vertebral joint. The nerve root is located approximately 1 cm below the plane of the transverse process in the so called intertransverse space. In order to be able to reach the nerve root by surgery, it must be possible to uncover the adjacent transverse processes and the vertebral joint. Because the vertebral joint is located approximately 2 cm above the plane of the transverse process, the medial valve of the retractor is advantageously approximately 2 cm shorter than the cranial, caudal and lateral valve. Advantageously, the cranial and caudal valve is applied to the transverse processes. Subsequently, the surgical instruments, particularly an endoscope, are attached to the slide and introduced into the surgical space.

An embodiment example of the invention is further explained with reference to the drawings in the appendix.

FIG. 1 shows a top view of the carrier ring of the retractor,

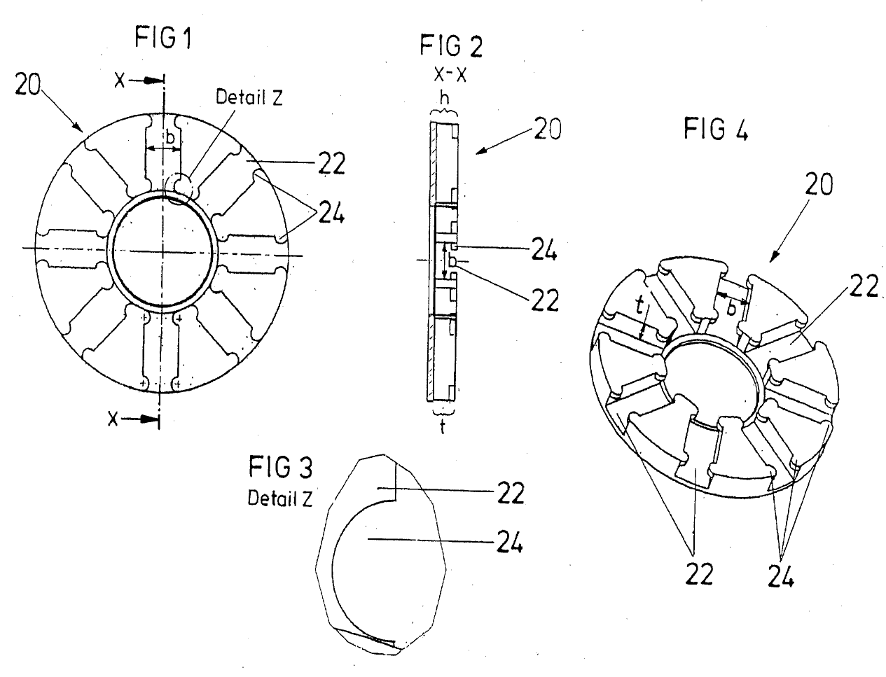

FIG. 2 shows a diametric cross section through the carrier ring of the retractor along the line X-X in FIG. 1,

FIG. 3 shows an enlargement of a detail of FIG. 1,

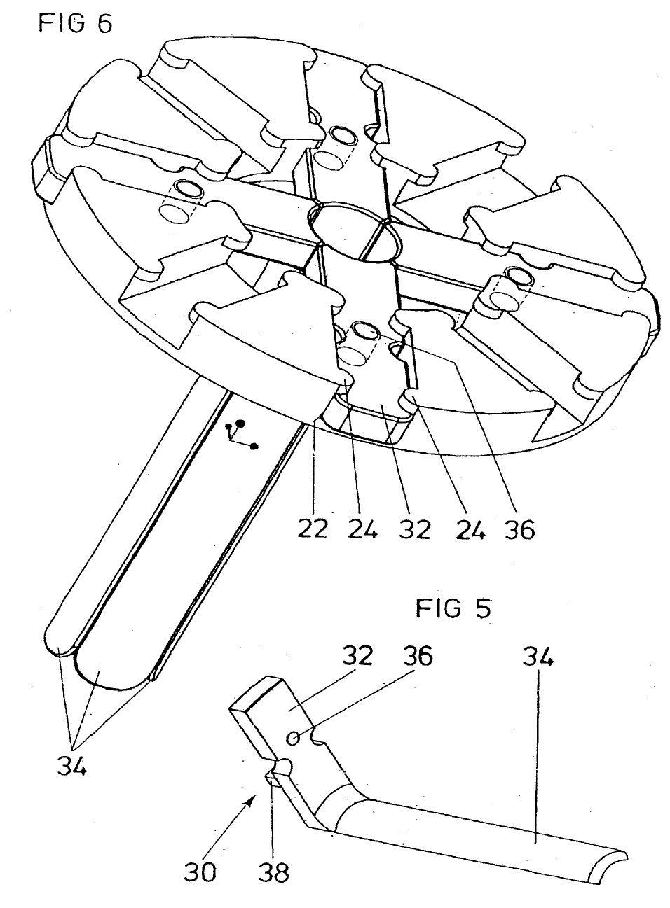

FIG. 4 shows a perspective view of the carrier ring of the retractor,

FIG. 5 is a perspective view of a valve,

FIG. 6 is a perspective view of the carrier ring of the retractor with inserted valves in the telescoped state,

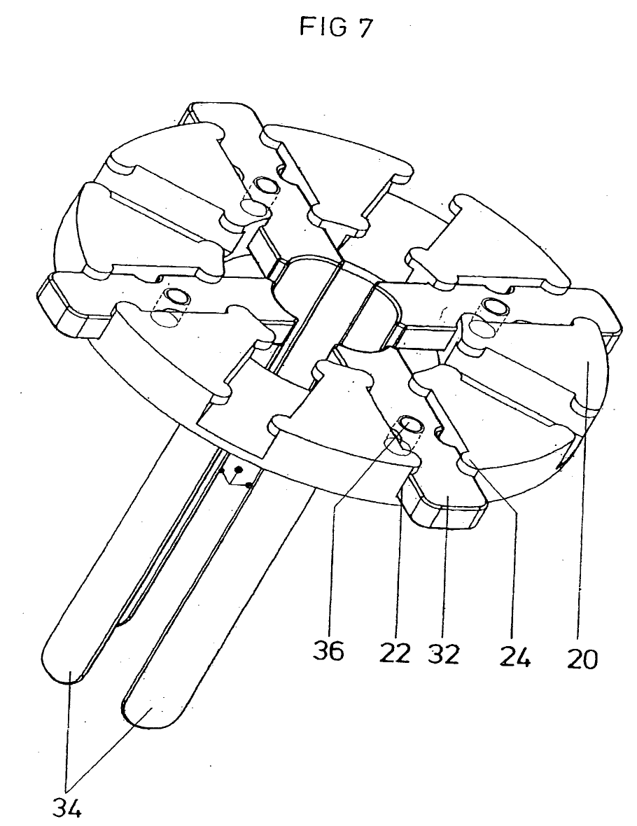

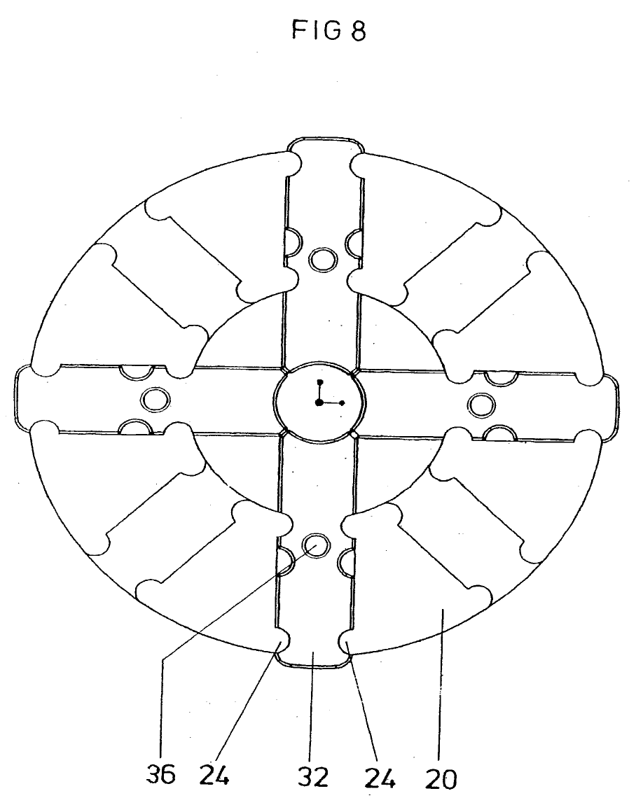

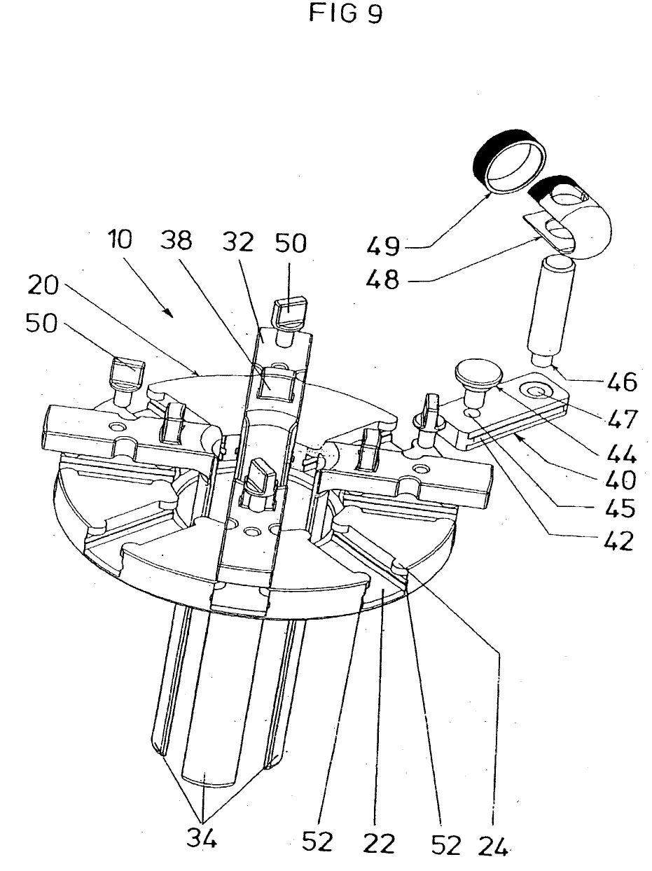

FIG. 7 shows a perspective view of the carrier ring with inserted valve in the expanded state,

FIG. 8 shows a top view of the carrier ring with inserted valves in the telescoped state,

FIG. 9 is an exploded view of the retractor,

FIG. 10 shows the representation of the access route to the site of the lesion,

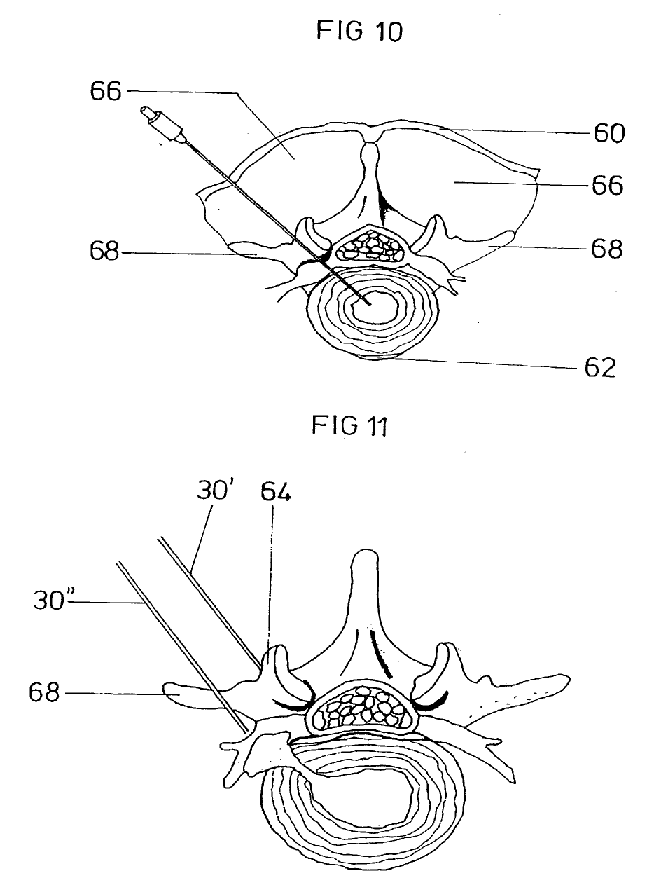

FIG. 11 shows a representation of the surgical field with introduced retractor (transverse cross section), and

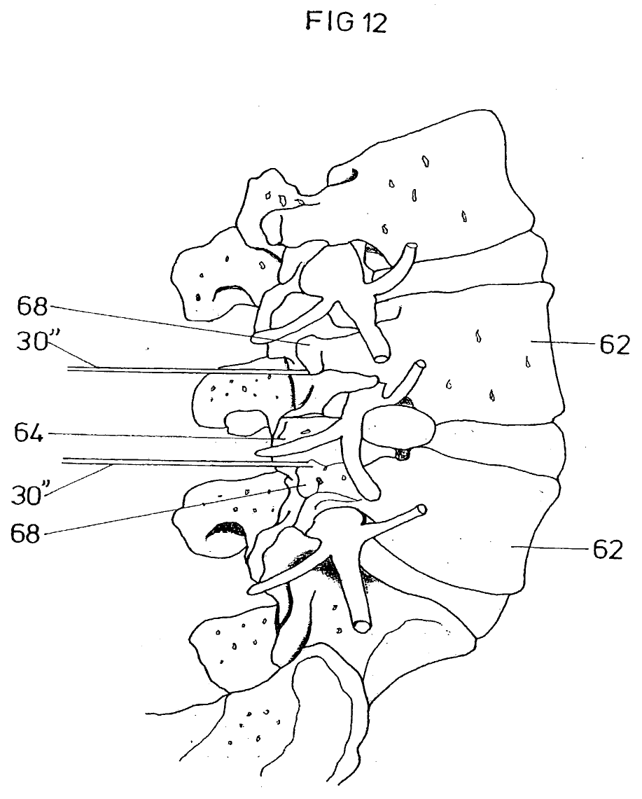

FIG. 12 shows a representation of the surgical field with introduced retractor (sagittal cross section).

FIGS. 1-4 show different views of a carrier ring for a retractor 10. The carrier ring 20 presents eight radial guide grooves 22, which are mutually separated by an angle of 45° and whose width b is constant over the entire radial length of the guide nut 22. The carrier ring 20 presents an external diameter dₐ and an internal diameter dᵢ. The external diameter dₐ in this embodiment example is 9.9 cm and the internal diameter dᵢ is 3.9 cm. The carrier ring 20 presents a height h of approximately 1 cm, where the depth t of the guide grooves 22 is approximately 3/4 of the height h of the carrier ring 20. The width b of the guide grooves 22 is approximately 1.4 cm. Each guide groove 22 presents 4 semicircular projections 24, which are arranged at the upper edge of each guide nut 22 in the plane of the carrier ring 22 and which extend into the guide nut 22. As a result, an arm 32 of a valve 30 which is introduced into the guide nut 22 is prevented by application from falling out or from being pushed out upward during the placement of the retractor in the surgical field. Naturally, other types of securing are also possible. FIG. 9, in addition, shows grooves 22 which run parallel to the bottom of the guide grooves 22 at mid-height of the guide groove 22, in which a flange or similar part of the arms 32 of the valves 30 can be guided.

FIG. 5 shows one of the L-shaped valves 30, which are inserted with the arm 32 in one of the guide grooves 22 of the carrier ring 20. The second arm 34 of the valves 30 extends out of the carrier ring 20 on the side turned away from the guide grooves 22. The arms 34 of the valves 20 present a convex curvature toward the external margin of the carrier ring. The arch comprises an angular segment of 90° so that, at the time of the assembly of the arms 34 of the four valves 30, a complete tube is produced. In the arm 32, which is led in the guide nut 22, a perforating bore 36 is provided, into which a thumb screw 50 can be inserted to clamp and stop the valve 30 in the guide groove 22.

On the side of the arm 32 which is turned toward the carrier ring 20 in the inserted state of the valve 30, a bracket 38 can be attached, by means of which the valve 30 can easily be moved in the guide groove 22. The applied bracket 38 is approximately as broad as it is high and presents, in the radial direction of the carrier ring, a thickness of approximately 1 of the height of the bracket 38. Here the bracket, in cross section, has a convex curvature toward the external margin of the carrier ring 20, which results in the adaptation of the bracket 38 to the curvature of the tips of the fingers of the person who operates the retractor 10.

FIGS. 6-8 illustrate how the valves 30 can be inserted into the carrier ring 20. FIG. 6 shows the four valves 30 in the telescoped state. After the retractor 10 has been inserted in this state into the surgical space, the valves 30 are moved radially and outward in the guide grooves 22 resulting in the state indicated in FIG. 7. The surgical field which is enclosed by four arms 34 of the valves 30 has become considerably enlarged as a result of the shifting outward of the valves 30.

A representation of the retractor 10 with all components is shown in FIG. 9. In addition to the valves 30 and their attachment by means of thumb screws 50, the slide 40 is shown. The slide 40 is a substantially rectangular element having a width which corresponds to the width b of the guide grooves 22. On both longitudinal sides of the slide 40, two flanges 42 are applied, by means of which the slide 40 is guided in the grooves 52 of the guide grooves 22. The slide 40 presents a perforating bore 45, into which an knurled screw 44 can be inserted to stop this slide 40 by clamping in the guide groove 22. For this purpose, the bore 45 is provided with an internal threading. The slide 40 presents an additional bore 47 into which bolt 46 can be inserted. A chamber 48 can be applied onto the bolt 46, in which chamber an endoscope or another surgical instrument can be attached by means of an knurled nut 49.

All the described components of the retractor 10 present rounded edges to reduce the risk of damaging the tissue by the retractor 10. Moreover, all the components of the retractor 10 are made of titanium, which is biocompatible, and which presents good mechanical properties and a low weight.

The surgical treatment of the lateral lumbar intervertebral disk prolapse with the aid of the retractor 10 according to the invention is described below with a reference to FIGS. 10, 11 and 12. A paravertebral dorsal incision is made into the skin 60. The incision should not exceed a length of 4 cm. Starting from the incision, access to the site of the lesion is achieved through the dorsal muscles 66 in a ventral medial direction toward the transverse process 68 of the vertebrae which are adjacent to the intervertebral disk and the corresponding small vertebral joint 64. The length of the access route is, as a function of the thickness of the fatty layer under the skin 60 and the dorsal muscle 66, approximately 8-12 cm, so that the arms 34 of the retractor 10 must present a corresponding length.

The surgical space is delimited cranially and caudally by the transverse process 68 of the adjacent vertebrae 62 and medially by the vertebral joint 64. The nerve root to be treated is located approximately 1 cm below the plane of the transverse process 68 in the so called intertransverse space. The vertebral joint 64 is located approximately 2 cm above the plane of the transverse process 68 and, at first, it constitutes the medial limitation of the access to the surgical field. The arm 34 of the medial valve 30′ of the retractor 10 therefore must be shorter by approximately 2 cm than the cranial, caudal and lateral valve 30″.

The retractor 10, at first, is introduced with telescoped valves 30 through the incision in the skin and the dorsal muscle 66 to the area of the lesion. Then, the valves 30 are shifted away from each other in the radial direction on the carrier ring 20. The cranial and caudal valves 30″ are anchored in the transverse process 68. The small length of the medial valve 30′ of the retractor 10 has the advantage that an osseous resection can be avoided.

After the introduction of the retractor 10, slides 40 are provided in the free guide grooves 22. The different surgical instruments, in particular an endoscope, can now be attached to the slide and introduced into the surgical field.

List of Reference Numbers

10 Retractor

20 Carrier ring

22 Guide grooves

24 Projection

30 Valve

32 Arm

34 Arm

36 Bore

38 Bracket

40 Slide

42 Flange

44 Knurled screw

45 Bore

46 Bolt

47 Bore

48 Clamp

49 Knurled nut

50 Thumb screw

52 Grooves

60 Skin

62 Vertebrae

64 Vertebral joint

66 Dorsal muscles

68 Transverse processes

dᵢ Internal diameter

dₐ External diameter

b Width

t Depth

h Height

1. Retractor (1) with a carrier ring (20) and L-shaped valves (30) which, with a first arm (32) which is parallel to the carrier ring lane, is oriented perpendicularly with respect to the carrier ring plane, in a manner so it can be attached to the carrier ring (20) second arm (34) and which presents, in cross section, a convex curvature toward the external margin of the carrier ring (20), characterized in that the arm (32) of the valves (30) which is attached to the carrier ring (20) are guided on the carrier ring (20) in radially oriented guides (22).

2. Retractor according to claim 1,
characterized in that
the curvature of the second arm (34) of the valves (30) is designed such that during the shifting of the valves (30) radially and inward on the carrier ring (20), a complete tube is assembled.

3. Retractor according to one of the preceding claims,
characterized in that four valves (30) form a complete tube.

4. Retractor according to one of the preceding claims,
characterized in that each valve (30) can be fixed by means of a screw which can be adjusted by hand, for example a thumb screw (50).

5. Retractor according to one of the preceding claims,
characterized in that the valves (30), which are attached to the carrier ring (20), present a different length of the second arm (34).

6. Retractor according to one of the preceding claims,
characterized in that additional radial guides (22) are sunk in the carrier ring (20) in which the slide (40) can be inserted, to which surgical instruments such as endoscopes or similar devices can be attached.

7. Retractor according to claim 6,
characterized in that, on the slide (40) a bolt (46) is inserted vertically, to which a chamber (48) is attached by means of a nut, for example a knurled nut (49) in which chamber an endoscope or a similar device can be attached.

8. Retractor according to one of the preceding claims,
characterized in that on the carrier ring (20), a handle can be attached for positioning the retractor (10).

9. Retractor according to one of the preceding claims,
characterized in that all the components are made of titanium.
